(19) United States
(12) Patent Application Publication
(10) Pub. No.: US 2005/0287673 A1
(43) Pub. Date: Dec. 29, 2005

(54) Reactor mixing

(75) Inventors: Timothy J. Johhson, Andover, MA (US); A. Peter Russo, Woburn, MA (US); Brian O. Benoit, Woburn, MA (US); Andrey J. Zarur, Winchester, MA (US); Seth T. Rodgers, Somerville, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: BioProcessors Corp., Woburn, MA (US)

(21) Appl. No.: 11/147,413

(22) Filed: Jun. 7, 2005

Related U.S. Application Data
(60) Provisional application No. 60/577,986, filed on Jun. 7, 2004.

(51) Int. Cl. G01N 33/00

(52) U.S. Cl. 436/34

(57) ABSTRACT

Immiscible substances, such as gases, solids or liquids may be included within a reaction site container as a mixer of a liquid sample. Movement of the mixer within the container may help suspend or re-suspend cells or other species. Movement of the mixer also may generate shear forces that can affect cellular activity. In some embodiments, movement of the container brings about movement of the mixer. Containers may be mounted to a rotating apparatus in various orientations to achieve different travel paths of the mixer. Varying the rotation rate and/or the relative densities of the mixer and the liquid sample also may affect the mixer travel path.

### RELATED APPLICATIONS

This application claims priority under 35 U.S.C. § 119(e) to U.S. Provisional Application Ser. No. 60/577,986, entitled “Reactor Mixing”, filed on Jun. 7, 2004, which is herein incorporated by reference in its entirety.

### FIELD OF THE INVENTION

The present invention generally relates to chemical, biological, and/or biochemical reactor chips and/or reaction systems such as microreactor systems.

### DESCRIPTION OF THE RELATED ART

A wide variety of reaction systems are known for the production of products of chemical and/or biochemical reactions. Chemical plants involving catalysis, biochemical fermenters, pharmaceutical production plants, and a host of other systems are well-known. Biochemical processing may involve the use of a live microorganism (e.g., cells) to produce a substance of interest.

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

As described in International Patent Application Serial No. PCT/US01/07679, published on Sep. 20, 2001 as WO 01/68257, entitled “Microreactors,” incorporated herein by reference, cells have also been cultured on a very small scale (i.e., on the order of a few milliliters or less), so that, among other things, many cultures can be performed in parallel.

While important and valuable advances have been made in cell culturing and other fields, improvements would be valuable.

### SUMMARY OF THE INVENTION

Each of the following commonly-owned applications directed to related subject matter and/or disclosing methods and/or devices and/or materials useful or potentially useful for the practice of the present invention is incorporated herein by reference: U.S. patent application Ser. No. 10/457,017, filed Jun. 5, 2003, entitled “System and Method for Process Automation,” by Rodgers, et al.; U.S. patent application Ser. No. 10/457,049, filed Jun. 5, 2003, entitled “Materials and Reactor Systems having Humidity and Gas Control,” by Rodgers, et al, published as 2004/0058437 on Mar. 25, 2004; U.S. patent application Ser. No. 10/457,015, filed Jun. 5, 2003, entitled “Reactor Systems Having a Light-Interacting Component,” by Miller, et al., published as 2004/0058407 on Mar. 25, 2004; U.S. patent application Ser. No. 10/456,929, filed Jun. 5, 2003, entitled “Apparatus and Method for Manipulating Substrates,” by Zarur, et al.; U.S. patent application Ser. No. 10/664,046, filed Sep. 16, 2003, entitled “Determination and/or Control of Reactor Environmental Conditions,” by Miller, et al., published as 2004/0132166 on Jul. 8, 2004; U.S. patent application Ser. No. 10/664,068, filed Sep. 16, 2003, entitled “Systems and Methods for Control of pH and Other Reactor Environmental Conditions,” by Miller, et al., published as 2005/0026134 on Feb. 3, 2005; U.S. patent application Ser. No. 10/664,067 filed on Sep. 16, 2003, entitled “Microreactor Architecture and Methods,” by Rodgers, et al.; and U.S. Patent Application Ser. No. 60/577,985 filed on Jun. 7, 2004, entitled “Control of Reactor Environmental Conditions,” by Rodgers, et al. The present invention generally relates to chemical, biological, and/or biochemical reactor chips and/or reaction systems such as microreactor systems. The subject matter of this invention involves, in some cases, interrelated products, alternative solutions to a particular problem, and/or a plurality of different uses of one or more systems and/or articles.

According to one embodiment of the invention, a method includes introducing a liquid sample into a reaction site container having a volume of less than about 2 mL and comprising a detection region. The method also includes moving a mixer within the liquid sample to mix the liquid, wherein the mixer is freely movable within the container and able to move into the detection region. The method further includes moving the mixer outside of the detection region, and detecting a property of the liquid present in the detection region.

In some embodiments, the reaction site container may be constructed and arranged to maintain at least one living cell. In some embodiments, a gas permeable, liquid vapor impermeable membrane may define a first wall of the container.

According to another embodiment of the invention, an apparatus includes a chemical, biological, or biochemical reactor chip comprising a reaction site container having a volume of less than about 2 mL, the container comprising a detection region the reactor chip also includes a volume of a liquid sample within the container, a mixer for mixing the liquid sample, the mixer freely movable within the container in at least one container orientation, and an impediment within the container constructed and arranged to limit the presence of the mixer within the detection region.

In some embodiments the chip is able to maintain at least one living cell. In some embodiments, the at least one living cell is mammalian. Optionally, in certain embodiments, the reactor chip may further include a gas permeable, liquid vapor impermeable membrane that defines a first wall of the container.

According to another embodiment of the invention, a method includes introducing a liquid sample into a reaction site container having a volume of less than about 2 mL, the reaction site container comprising a detection region and an impediment within the reaction site container. The method also includes orienting the container in a first orientation that causes the mixer to move within the detection region to mix the liquid, orienting the container in a second orientation that causes the mixer to move outside of the detection region, orienting the container into a detection orientation in which the mixer is impeded from moving into the detection region by the impediment, and detecting a property of the liquid present in the detection region.

Other advantages and novel features of the invention will become apparent from the following detailed description of the various non-limiting embodiments of the invention when considered in conjunction with the accompanying figures. In cases where the present specification and a document incorporated by reference include conflicting and/or inconsistent disclosure, the present specification shall control. If two (or more) applications incorporated by reference include conflicting and/or inconsistent disclosure with respect to each other, then the later-filed application shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates a layer of a chip including six reactors including reaction site containers that can be used in accordance with one embodiment of the invention;

FIGS. 2a-2c illustrate various orientations in which chips may be positioned on a rotating apparatus;

FIGS. 3a-3c show selected movement directions of immiscible substances within containers;

FIG. 4 shows one illustrative embodiment of a rotating apparatus that can be used in accordance with the invention;

FIG. 5a illustrates a top view of a reaction site container having a gas bubble impediment according to one embodiment of the invention;

FIG. 5b illustrates a cross-sectional side view of the embodiment shown in FIG. 5a; and

FIG. 6 illustrates a chip oriented and rotated on a rotating apparatus such that the position of a gas bubble is controlled.

### DETAILED DESCRIPTION

The present invention generally relates to chemical, biological, and/or biochemical reactor chips and/or reaction systems such as microreactor systems, as well as systems and methods for constructing and using such devices. In one aspect of the invention, a chip, reactor, or a reaction system containing a liquid sample may be configured for mixing with a mixer, such as a gas bubble, a glass bead, or a liquid that is immiscible with the liquid sample. The invention involves control over the location of the mixer, for example to allow the mixer to effectively mix when desired, and such that its presence is limited within the detection region, e.g., it can be kept separate from a detection region of the chip or reactor during certain operations, such as detection, measurement or sensing operations, so as to not interfere with these operations. Detection of properties of a liquid or other substance within the chip or reactor, or environmental conditions within the chip or reactor can be performed once it has been determined that the mixer is not located within the detection region of the chip or reactor. In some embodiments of the invention, an apparatus revolves and/or rotates chips to move a mixer within a chip.

In one embodiment, mixers are restrained from being present within a detection region of a chip or reactor. In certain embodiments, impediments such as physical barriers may be used to contain gas bubbles that act as mixers within a gas containing region, or otherwise away from a detection region.

Chip or reaction systems used in accordance with the invention may include reaction sites that can be very small, for example, having a volume of less than about 2 milliliters. In some embodiments, the reaction site includes compartments or containers that include a surface that is formed with a membrane. In certain embodiments, the chips or other reaction systems include one or more reaction sites or reaction site containers.

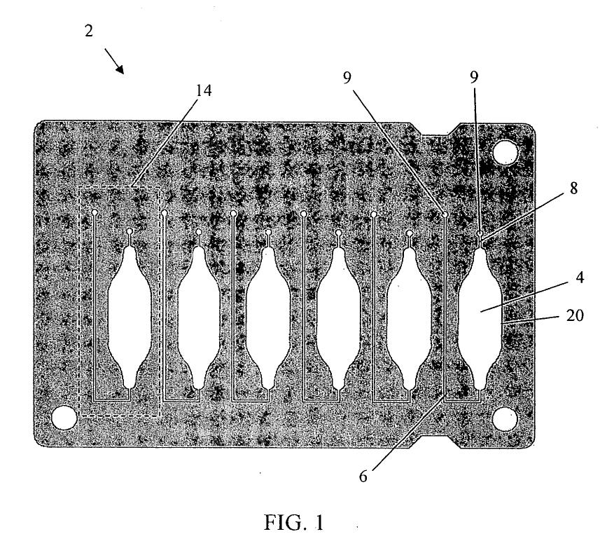

Referring now to FIG. 1, one portion of a chip according to one embodiment is illustrated schematically. The portion illustrated is a layer 2 which includes within it a series of void spaces which, when layer 2 is positioned between two adjacent layers (not shown), define a series of enclosed channels and reaction sites.

FIG. 1 represents an embodiment including six reaction sites 4 defined by reaction site containers 20. Reaction sites 4 define a series of generally aligned, elongated voids within a relatively thin, generally planar piece of material defining layer 2. Reaction sites 4 can be addressed by a series of channels including channels 8 for delivering species to reaction sites 4. In FIG. 1, each reaction site 4, along with the associated fluidic connections (e.g., channels 6 and 8, and ports 9), together define a reactor 14, as indicated by dashed lines. In FIG. 1, layer 2 contains six such reactors, each reactor having substantially the same configuration. In other embodiments, a reactor may include more than one reaction site, and/or additional channels, ports, etc. A chip can include any number of reactors, any or all of which can be identical, or any of which can be different (e.g., different sized containers, different shaped containers, different set of access channels, etc).

An immiscible substance may be provided in reaction site container 20 to act as a mixer. By moving an immiscible substance within reaction site container 20, the liquid sample and/or solids suspended in the liquid may be agitated. In some embodiments, an immiscible substance may be moved by introducing an immiscible substance having a density that is different from the average density of the liquid sample or carrier liquid and changing the orientation of the container. This density difference can be, for example, at least 1% different than the average density of the liquid sample or carrier liquid, at least 2% different, 5%, 7%, or 10% or more different. The change in orientation causes the immiscible substance of different density to rise or sink within reaction site container 20 depending on whether the immiscible substance has a higher or lower density than the liquid sample.

As used here in, “immiscible” defines a relationship between two substances that are largely immiscible with respect to each other, but can be partially miscible. “Immiscible” substances, even if somewhat miscible with each other, will largely remain separate from each other in an observable division. For example, air and water meet this definition, in that a container of the invention containing primarily water or an aqueous solution and some air will largely phase-separate into an aqueous portion and a gas bubble or gas region, even though air is slightly soluble in water and water vapor may be present in the air. Other examples of immiscible substances, albeit those that may be somewhat miscible with each other, include oil and water, polymeric bead and water, and the like. Those of ordinary skill in the art will understand, from this definition, and the description that follows involving techniques for managing immiscible substances, the meaning of this term.

The introduction of an immiscible substance within container 20 may include the addition or creation of a gas bubble. The gas bubble may be introduced by partially filling the container with a liquid sample and leaving a portion as the originally present gas (typically air). In other embodiments, evaporation or cellular respiration may form a gas bubble. Solid substances, such as polymeric or glass beads may be included in container 20 to act as mixers. It is also possible to use a liquid that is immiscible with the liquid sample as a mixer. Of course any combination of the above immiscible substances also may be used within a container.

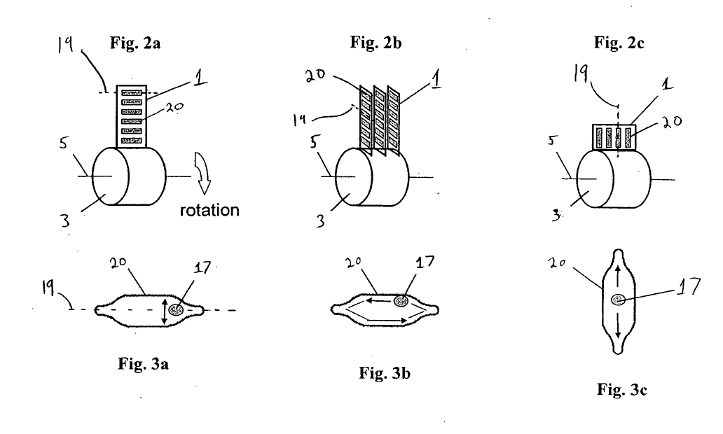

As shown in FIGS. 2a-2c, chips 1 including reaction site container 20 may be mounted to a rotating apparatus 3. When rotating apparatus 3 rotates, the orientation of chip 1 relative to gravity changes and immiscible substances of different densities move relative to one another within reaction site container 20. FIG. 2a shows a radial mounting orientation for a chip containing six reaction site containers 20. As chip 1 revolves around axis 5 via the rotation of rotating apparatus 5, an immiscible substance 17 moves up and down relative to gravity which results in lateral movement within reaction site container 20, as shown in FIG. 3a. Immiscible substance 17 may reach the side walls of reaction site container 20 depending-on the rotation rate and the relative densities of immiscible substance 17 and the liquid sample. At high rotation rates, immiscible substance 17 does not have time to move entirely to one side wall before reaction site container 20 is reversed relative to buoyancy or gravitational forces, and immiscible substance 17 moves in the opposite direction. At slower rotation rates or higher density differences, immiscible substance 17 moves faster and may reach one side wall before the reaction site container orientation is reversed.

FIG. 2b shows a vertical mounting orientation for three chips 1 on rotating apparatus 3. In this embodiment, immiscible substance 17 tends to follow a circuitous path within container 20 when chip 1 is revolved around axis 3, as shown in FIG. 3b. Such a path may help re-suspend cells or other species that have attached or settled along the inside perimeter of container 20. Similar to the embodiment of FIG. 2a, the extent of travel of immiscible substance 17 depends on the rotation rate and the relative densities of immiscible substance 17 and the liquid sample.

FIG. 2c shows a horizontal orientation for mounting chip 1 on rotating apparatus 3. In this orientation, immiscible substance 17 moves in an end-to-end direction during rotation. Similar to the embodiments of FIGS. 2a and 2b, the extent of travel of immiscible substance 17 depends on the rotation rate and the relative densities of immiscible substance 17 and the liquid sample.

The apparatuses described may be configured to secure the chip, article, or other substrate in any of a variety of suitable orientations. Depending on the configuration of the chip, article, or other substrate, certain such orientations may be particularly advantageous for imparting a desired degree or pattern of mixing or agitation. As explained in more detail below in the context of FIGS. 2a-2c, this can be important for manipulation of articles comprising one or a plurality of elongate containers.

“Elongate,” as used herein when referring to a chamber or substrate or container or predetermined reaction site of an article, refers to such chamber or substrate or container or predetermined reaction site having a perimetric shape, e.g. of an outer boundary or container, that is characterized by there being a first straight line segment, contained within the outer boundary/container, connecting two points on the outer boundary/container and passing through the geometric center of the chamber or substrate or container or predetermined reaction site that is substantially longer than a second straight line segment, perpendicular to the first line segment, contained within the outer boundary/container, connecting two points on the outer boundary/container—other than the same two points connected by the first line segment—and passing through the geometric center of the chamber or substrate or container or predetermined reaction site. For example, if the article is a planar chip comprising a volumetric container defining a predetermined reaction site characterized by a thickness, measured in a direction perpendicular the plane of the chip and a length and width, measured in mutually perpendicular directions both parallel to the plane of the chip, the predetermined reaction site would be “elongate,” if the length substantially exceeded the width (e.g. as would be the case for a thin, rectangular or ellipsoidal, tear-shaped, etc., predetermined reaction site). An axis co-linear with the longest such straight line segment, contained within the outer boundary/container, connecting two points on the outer boundary/container and passing through the geometric center of the chamber or substrate or container or predetermined reaction site for an elongate chamber, substrate, container or predetermined reaction site is referred to herein as the “longitudinal axis” of the chamber or substrate or container or predetermined reaction site.

For example, in FIG. 2a, a chip 1, comprising a plurality of elongate containers 20, such as biological containers (for example, defining a predetermined reaction site), each characterized by a longitudinal axis 19, is secured to rotating apparatus 3 configured to revolve the article about a substantially horizontal axis 5. Chip 1 is secured to apparatus 3 such that the longitudinal axes 19 of containers 20 are arranged with respect substantially horizontal axis 5 so that longitudinal axes 19 are parallel to horizontal axis 5. In a preferred arrangement, shown in FIG. 2b, chips 1 are secured to apparatus 3 such that the longitudinal axes 19 of containers 20 are arranged with respect to substantially horizontal axis 5 so that longitudinal axes 19 are perpendicular to and non-intersecting with substantially horizontal axis 5. In the configuration illustrated in FIG. 2c, chip 1 is secured to apparatus 3 such that the longitudinal axes 19 of containers 20 are arranged with respect substantially horizontal axis 5 so that longitudinal axes 19 are perpendicular to and intersect with substantially horizontal axis 5.

Rotating apparatus 3 may be rotated at any suitable rate. In some embodiments, rotation rates of 4 rpm, 8 rpm or 12 rpm are used, for example. In other embodiments, much higher or much lower rotation rates would be suitable depending on the species present in the liquid sample, the type and density of mixer present, the size of the container and the rotation apparatus, and other factors.

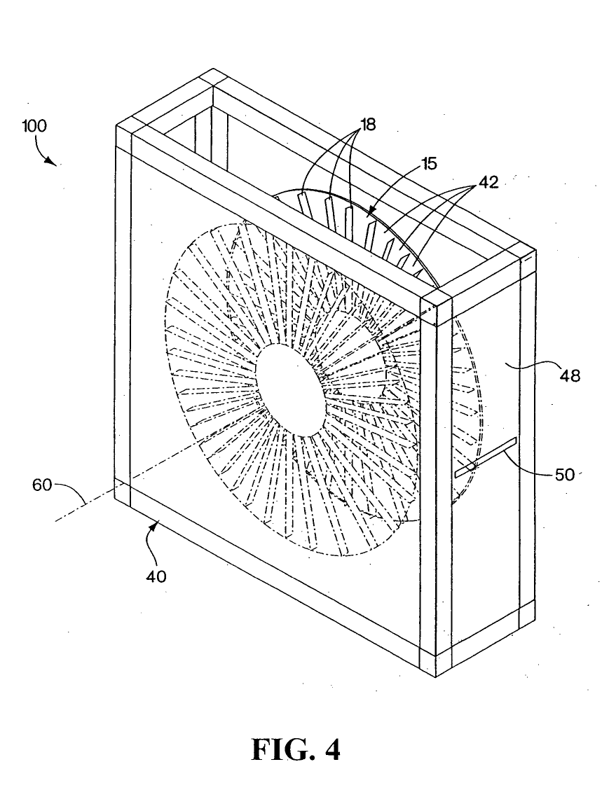

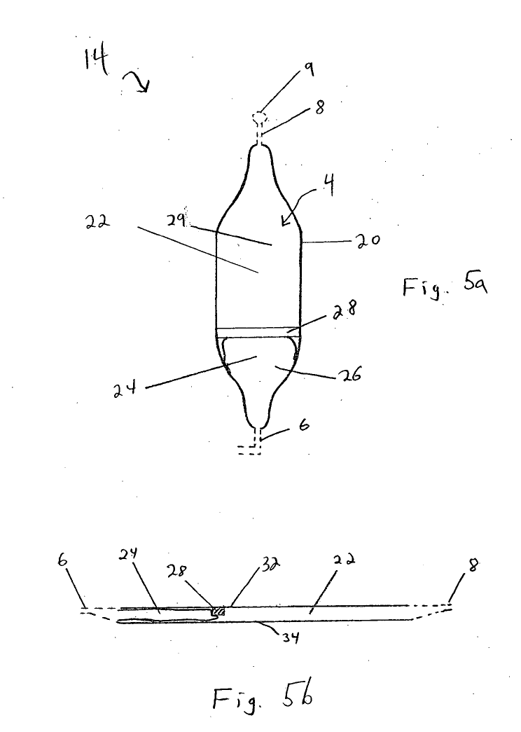

FIG. 4 shows an apparatus 100 for manipulating a chemical, biological, or biochemical sample in accordance with a variety of embodiments of the present invention. Apparatus 100, and other arrangements shown in the figures, are intended to be exemplary only. Other arrangements are possible and are embraced by the present invention. Apparatus 100 includes a housing 40 of generally rectangular solid shape (although the apparatus itself is not solid). In the embodiment illustrated housing 40, apparatus 100 includes two, generally square, opposed major surfaces joined by four edges of rectangular shape. Housing 40 may be, for example, an incubator. In some cases, housing 40 may be sufficiently enclosed so as to keep device 15 clean, free of dust particles, within a laminar flow field, sterile, etc., depending on the application.

Mounted within housing 40, on an axis 60 passing through the two, opposed major surfaces of the housing, is a device 15 for securing a plurality of individual substrates such as chips (not shown in FIG. 4) which may be constructed to contain a sample. Device 15 takes the form of a rotatable wheel with a plurality of radially outwardly extending members 18 which define, therebetween, a plurality of slots 42 within which one or more chips can be positioned. Once the chips are secured within slots 42, device 15 can be rotated, manually or automatically, about axis 60, thereby periodically inverting the chips secured in slots 42. Of course, in some embodiments, axis 60 may pass through only one of the major surfaces of the housing.

Within one face 48 of housing 40, which defines one of the edges of the housing joining the opposed major surfaces, is access port 50 through which a chip (or other substrate) can be introduced into and removed from the interior of housing 40. Access port 50 may be positioned anywhere within housing 40 that allows suitable access of chips or other substrates to apparatus 100, for example, in a side of housing 40 or on one or more major surfaces of housing 40. For the insertion of a chip into device 15 to be secured within a slot 42 of device 15, device 15 can be rotated so that a desired slot is aligned with access port 50, and a chip is inserted through access port 50 to be secured by a slot 42 within a selection region. Device 15 can be rotated to any predetermined radial orientation aligning a desired slot 42, with access to access port 50, so that one or more chips can be positioned within predetermined slots 42, and their location known so the chips can be removed from device 15 such that a predetermined slot securing a predetermined chip is aligned with access port 50 for external removal (for example, within a selection region). The chips (or other substrates) can be inserted into and removed from housing 40 via slot 50 by essentially any technique including manual operation by hand, operation by an actuator, or robotic actuation, as described more fully below. Access port 50 can be an opening in wall 48 of the housing, optionally including a flap, door, or other member that allows access port 50 to be closed when not being used to introduce or remove a chip from the housing. Additional arrangements are described below.

According to one aspect of the invention, the system is constructed and arranged to hinder the movement of mixers into a detection region of the reaction site containers. Chips of the invention can be constructed and arranged so as to be able to detect or determine one or more environmental conditions and/or sample properties associated with a reaction site of the chip or reactor, for example, by using a sensor. Many sensors, including optical sensors, make use of optical sensing equipment to measure environmental conditions or the presence of various substances contained in the reactor system. The presence of a mixer, such as a gas bubble or a glass bead, within the sensing area of the sensor can alter measurement results and lead to inaccuracies.

For example, as shown in FIGS. 5a and 5b, a reactor 14 comprises a container 20 that contains a liquid sample 22 and a gas bubble 24. Gas bubble 24 is shown in FIGS. 5a and 5b as being contained within a gas containing region 26. An impediment in the form of a physical barrier impedes the movement of gas bubble 24 out of gas containing region 26 and toward reaction site 4, which may contain detection region 29. In this embodiment, the physical barrier is a protrusion 28 which extends approximately halfway from a top interior surface 32 of reaction site container 20 to a bottom interior surface 34. When reaction site 4 is held substantially horizontally, protrusion 28 impedes the movement of gas bubble 24.

In some embodiments, to move the gas bubbles away from detection region 29, container 20 may be tilted away from horizontal for a sufficient length of time so that the buoyant forces on the gas bubbles move them into gas containing region 26 where they combine with gas bubble 24. Upon returning reaction site container 20 to a substantially horizontal position, movement of gas bubble 24 is impeded by protrusion 28.

In other embodiments, instead of using impediments to restrain an immiscible substance from moving into a detection region, a chip may be removed from a rotating apparatus or other holding apparatus and oriented to move the immiscible substance into a region outside of the detection region(s). For example, if a gas bubble is introduced as a mixer, holding the container at a slight angle relative to horizontal may be adequate to move the gas bubble to one of the container and out of any detection region(s). Once detection operation(s) are completed, the container may be returned to its holding apparatus.

In some embodiments, certain orientations of container 20 during rotation of chip 1 on rotating apparatus 3 result in control of immiscible substance 17 such that it is outside of the detection region. In such an embodiment, chip 1 may be temporarily held in an orientation that moves immiscible substance 17 outside of the detection region to permit a detection operation. Alternatively, a detection operation may be performed when chip 1 is in such an orientation although rotation is not halted or slowed.

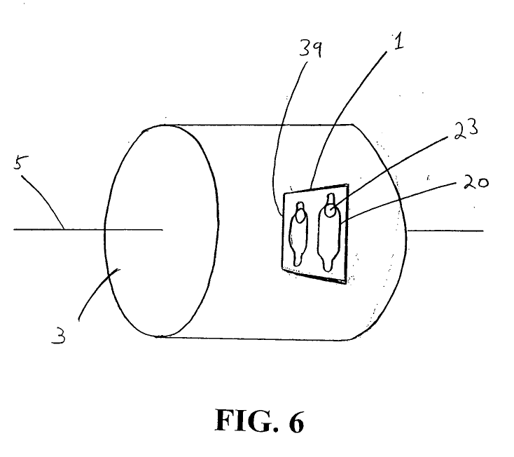

For example, as shown in FIG. 6, chip 1 is vertically mounted to rotating apparatus 3 at a near end 39 of chip 1 (an arrangement similar to the one shown in FIG. 2b), and rotating apparatus 3 is rotated such that an immiscible substance, such as a gas bubble 23, floats to one end of container 20. In this orientation, environmental conditions or liquid sample properties may be detected by sensing regions of container 20 that are separate from the upper end of container 20.

Instead of, or in addition to, moving container 20 to move immiscible substance 17 out of detection region 29, a magnetic, electrical, ceritrifugal, or other force may be applied to the container to contain the immiscible substance 17 so that it is maintained out of detection region 29, and/or to move the immiscible substance 17 from the detection region.

A “chemical, biological, or biochemical reactor chip,” (also referred to, equivalently, simply as a “chip”) as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

In some embodiments, two or more components of the chip may be joined using an adhesive material. As used herein, an “adhesive material” is given its ordinary meaning as used in the art, i.e., an auxiliary material able to fasten or join two other materials together. For instance, an adhesive may be used to bind a membrane to a substrate layer defining a reaction site. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid. For example, in certain embodiments, the adhesive may be applied to the component(s) using transfer tape (e.g., a tape having adhesive material attached thereto, such that, when the tape is applied to the component, the adhesive, or at least a portion of the adhesive, remains attached to the component when the tape is removed from the component). In one set of embodiments, the adhesive may be a pressure-sensitive adhesive, i.e., the material is not normally or substantially adhesive, but becomes adhesive and/or increases its adhesive strength under the influence of pressure, for example, a pressure greater than about 6 atm or about 13 atm (about 100 psi or about 200 psi). Non-limiting examples of pressure-sensitive adhesives include AR Clad 7876 (available from Adhesives Research, Inc., Glen Rock, Pa.) and Trans-Sil Silicone PSA NT-1001 (available from Dielectric Polymers, Holyoke, Mass.).

In some embodiments, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous, for instance, when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. For instance, in one embodiment, an impediment is formed in an adhesive material positioned in a reaction site container of a chip. The impediment may be in contact with one or more interior surfaces of the container. Of course, other components of the chip may be constructed without the use of adhesive materials, as previously discussed.

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

As used herein, a “reaction site” is defined as a site within a reactor that is constructed and arranged to produce a physical, chemical, biochemical, and/or biological reaction during use of the chip or reactor. More than one reaction site may be present within a reactor or a chip in some cases, for example, at least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a substrate. The reaction site may be defined as a region where a reaction is allowed to occur; for example, a reactor may be constructed and arranged to cause a reaction within a channel, one or more compartments, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the chip or reactor, for example, a cytophilic region within the chip or reactor.

The term “detection region,” as used herein, generally refers to a region of the chip or reactor where sensors may be used to detect or determine environmental conditions and/or liquid sample properties. For example, a region of an upper layer and/or a bottom layer of a chip may be substantially transparent or semi-transparent such that optical measurements of substance contained within the chip may be acquired. In some embodiments, the detection region is contained within a reaction site container so that measurements may be made without moving the substances from the reaction site container or other reaction site.

The volume of the reaction site can be very small in certain embodiments and may have any convenient size. Specifically, the reaction site may have a volume of less than one liter, less than about 100 ml, less than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. In another set of embodiments, the reaction site may have a dimension that is 2 millimeters deep or less, 500 microns deep or less, 200 microns deep or less, or 100 microns deep or less.

In some embodiments of the invention, a reactor and/or a reaction site within a chip may be constructed and arranged to maintain an environment that promotes the growth of one or more types of living cells, for example, simultaneously. In some cases, the reaction site may be provided with fluid flow, oxygen, nutrient distribution, etc., conditions that are similar to those found in living tissue, for example, tissue that the cells originate from. Thus, the chip may be able to provide conditions that are closer to in vivo than those provided by batch culture systems. In embodiments where one or more cells are used in the reaction site, the cells may be any cell or cell type, for instance a prokaryotic cell (e.g., a bacterial cell) or a eukaryotic cell (e.g., a mammalian cell). The precise environmental conditions necessary in the reaction site for a specific cell type or types may be determined by those of ordinary skill in the art.

As used herein, a “membrane” is a thin sheet of material, typically having a shape such that one of the dimensions is substantially smaller than the other dimensions, that is permeable to at least one substance in an environment to which it is or can be exposed. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. Non-limiting examples of substances to which the membrane may be permeable to include water, O₂, CO₂, or the like. As an example, a membrane may have a permeability to water of less than about 1000 (g micrometer/m² day), 900 (g micrometer/m² day), 800 (g micrometer/m² day), 600 (g micrometer/m² day) or less; the actual permeability of water through the membrane may also be a function of the relative humidity in some cases.

Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or may allow water vapor to permeate across it, but at a rate that is at least an order of magnitude less than that for oxygen. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes may be transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent. The membrane, in one embodiment, may be used to divide a reaction site constructed and arranged to support cell culture from a second portion, for example, a reservoir. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. One or more membranes may also define one or more walls of a reaction site container. For instance, in one embodiment, a first membrane (e.g., a gas permeable vapor impermeable membrane) defines a first wall of a reaction site container. In another embodiment, a second membrane (e.g., a gas permeable vapor impermeable membranes) defines a second wall of the reaction site container. Of course, those of ordinary skill in the art will be able to design other arrangements, having varying numbers of cell culture portions, reservoir portions, and the like, as described herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one act, the order of the acts of the method is not necessarily limited to the order in which the acts of the method are recited.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising acts of:
(a) introducing a liquid sample into a reaction site container having a volume of less than about 2 mL and comprising a detection region;
(b) moving a mixer within the liquid sample to mix the liquid, wherein the mixer is freely movable within the container and able to move into the detection region;
(c) moving the mixer outside of the detection region; and
(d) detecting a property of the liquid present in the detection region.

2. A method as in claim 1, wherein the reaction site container is constructed and arranged to maintain at least one living cell.

3. A method as in claim 1, wherein a first gas permeable, liquid vapor impermeable membrane defines a first wall of the container.

4. A method as in claim 1, wherein the mixer is a solid.

5. A method as in claim 1, wherein the mixer is a gas.

6. A method as in claim 1, wherein the mixer is a liquid that is immiscible with the liquid sample.

7. A method as in claim 1, wherein the mixer has a density that is different from the average density of the liquid sample by at least 1%.

8. A method as in claim 1, wherein (d) comprises detecting a property of the liquid while the reaction site container is in a substantially horizontal position.

9. A method as in claim 1, wherein (d) comprises detecting a property of the liquid while the reaction site container is in a substantially vertical position.

10. A method as in claim 1, wherein (c) comprises orienting the container so that the mixer moves to a region outside of the detection region.

11. A method as in claim 10, wherein gravity moves the mixer to a region outside of the detection region.

12. A method as in claim 10, wherein buoyancy moves the mixer to a region outside of the detection region.

13. A method as in claim 10, wherein centrifugal force moves the mixer to a region outside of the detection region.

14. A method as in claim 1, wherein (c) comprises applying a force to the mixer so that the mixer moves to a region outside of the detection region.

15. A method as in claim 14, wherein (c) comprises applying a magnetic force to the mixer.

16. A method as in claim 5 wherein (c) comprises orienting the container so that the mixer moves to a region outside of the detection region.

17. A method as in claim 16, wherein the mixer moves to a gas containing region.

18. A method as in claim 5, wherein moving the gas outside of the detection region comprises moving the gas into a predetermined gas region in fluid communication with the reaction site container.

19. A method as in claim 1, wherein (c) comprises determining the location of the mixer.

20. A method as in claim 19, wherein (d) comprises detecting the property of the liquid in a region exclusive of the location of the mixer.

21. A method as in claim 1, wherein (c) further comprises determining whether the mixer is present within the detection region.

22. A method as in claim 1, wherein (b) comprises revolving the container around an axis that does not pass through the container.

23. A method as in claim 22, wherein revolving the container comprises rotating an apparatus to which the container is attached.

24. A method as in clam 23, wherein the container is attached to the apparatus in a substantially radial orientation.

25. A method as in clam 23, wherein the container is attached to the apparatus in a substantially vertical orientation.

26. A method as in clam 23, wherein the container is attached to the apparatus in a substantially horizontal orientation.

27. A method as in claim 1, wherein the liquid sample comprises dissolved species.

28. A method as in claim 1, wherein the liquid sample comprises suspended species.

29. A method as in claim 28, wherein the suspended species comprises cells.

30. A method as in claim 1, further comprising performing (a) through (d) for a plurality of reactors contained on a chemical, biological, or biochemical reactor chip.

31. A method as in claim 1, further comprising impeding the movement of a substance toward the detection region in the presence of a different, immiscible substance.

32. A method as in claim 5, further comprising:
(e) impeding movement of the gas into the detection region.

33. A method as in claim 32, wherein (e) comprises positioning a physical barrier within the container.

34. A method as in claim 1, wherein (b) comprises rotating the container around an axis that passes through the container.

35. A method as in claim 1, wherein introducing a liquid into the chip comprises accessing the inlet port via penetrating a self-sealing elastomeric material defining a portion of the inlet port.

36. A method as in claim 1, further comprising an inlet port and an outlet port, each in fluid communication with the reaction site container.

37. An apparatus comprising:
a chemical, biological, or biochemical reactor chip comprising a reaction site container having a volume of less than about 2 mL, the container comprising a detection region;
a volume of a liquid sample within the container;
a mixer for mixing the liquid sample, the mixer freely movable within the container in at least one container orientation; and
an impediment within the reaction site container constructed and arranged to limit the presence of the mixer within the detection region.

38. An apparatus as in claim 37, wherein the chip is able to maintain at least one living cell.

39. An apparatus as in claim 38, wherein the at least one living cell is mammalian.

40. An apparatus as in claim 37, further comprising a first gas permeable, liquid vapor impermeable membrane that defines a first wall of the container.

41. An apparatus as in claim 37, wherein the container has a volume of less than about 1 mL.

42. An apparatus as in claim 37, wherein the mixer is a gas bubble.

43. An apparatus as in claim 37, wherein the chip further comprises a predetermined gas region in fluid communication with the container.

44. An apparatus as in claim 43, wherein the mixer is positionable in the predetermined gas region when the mixer is not positioned in the detection region.

45. An apparatus as in claim 37, further comprising a self-sealing elastomeric material defining portions of the inlet and outlet ports.

46. An apparatus as in claim 37, wherein the container is defined by a void in a substrate layer.

47. An apparatus as in claim 46, wherein an adhesive layer binds the gas permeable, liquid vapor impermeable membrane to the substrate layer.

48. An apparatus as in claim 47, wherein the impediment is formed in the adhesive layer.

49. A method, comprising acts of:
(a) introducing a liquid sample into a reaction site container having a volume of less than about 2 mL, the reaction site container comprising a detection region and an impediment within the reaction site container;
(b) orienting the container in a first orientation that causes the mixer to move within the detection region to mix the liquid;
(c) orienting the container in a second orientation that causes the mixer to move outside of the detection region;
(d) orienting the container into a detection orientation in which the mixer is impeded from moving into the detection region by the impediment; and
(e) detecting a property of the liquid present in the detection region.

50. A method as in claim 49, wherein the reaction site container is constructed and arranged to maintain at least one living cell.

51. A method as in claim 49, wherein a first gas permeable, liquid vapor impermeable membrane defines a first wall of the reaction site container.

52. A method as in claim 51, where the reaction container further comprises a second gas permeable, liquid vapor impermeable membrane defining a second wall of the reaction site container.

53. A method as in claim 49, wherein the second orientation is a substantially vertical orientation.

54. A method as in claim 49, further comprising (f) orienting the reaction site container such that the mixer returns to the detection region.

55. A method as in claim 49, wherein the mixer is a gas bubble.

56. An apparatus as in claim 49, wherein the reaction site container is defined by a void in a substrate layer.

57. An apparatus as in claim 56, wherein an adhesive layer binds a gas permeable, liquid vapor impermeable membrane to the substrate layer.

58. An apparatus as in claim 50, wherein the impediment is formed in the adhesive layer.
